(19) United States
(12) Patent Application Publication
(10) Pub. No.: US 2003/0190272 A1
(43) Pub. Date: Oct. 9, 2003

(54) Sterilization containers and methods for radiation sterilization of liquid products

(76) Inventors: Dennis Raine, Plano, IL (US); Danette Bounelis, Yorkville, IL (US)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/210,707

(22) Filed: Aug. 1, 2002

Related U.S. Application Data
(60) Provisional application No. 60/370,666, filed on Apr. 8, 2002.

(51) Int. Cl. C23F 11/02; A61L 2/00

(52) U.S. Cl. 422/300; 422/297; 422/292

(57) ABSTRACT

The present invention provides, among other things, sterilization containers, inner bags, and methods for the radiation sterilization of liquid products. In particular, the invention provides sterilization containers for the irradiation sterilization of a pooled volume of liquid product greater than about, fifteen liters, and methods for the irradiation sterilization of liquid products.

### CROSS REFERENCE TO RELATED APPLICATIONS

This application claims the benefit of provisional U.S. patent application Ser. No. 60/370,666 filed Apr. 8, 2002.

### TECHNICAL FIELD

The present invention relates to sterilization containers, inner bags, and methods for radiation sterilization of liquid products. In particular, the invention preferably relates to sterilization containers for the sterilization of a pooled volume of liquid product greater than about fifteen liters, and to methods for sterilization by irradiation of liquid products.

### BACKGROUND

Liquid products for use in pharmaceutical, medical, research and biotech applications need to meet certain standards for sterility. Sterilization must be effective to inactivate or remove not only larger contaminating components present in the liquids, such as bacteria, mold, or fungus, but also must be effective to inactivate or remove smaller components, such as viruses, and potentially also bacteriophages. Inactivation or removal of these smaller components presents a particular challenge inasmuch as this must be done in a manner that does not damage (e.g., inactivate or render nonfunctional) certain larger molecules that also are present in the liquid products. These larger molecules include growth factors, antibodies, and other components that are necessary for the functioning or effectiveness of the liquid product for a particular application.

Sterilization by irradiation is a common method of sterilizing liquid products. Irradiation exposes products to radiant energy. The product is passed through an irradiator (i.e., an enclosed chamber) where it is exposed to a source of ionizing energy. The sources of ionizing energy can be gamma rays from cobalt 60 (⁶⁰Co), cesium 137 (¹³⁷Cs), x-rays, or electrons generated from machine sources. For gamma ray irradiation, the emitted gamma rays are very short wavelengths, similar to ultraviolet light and microwaves. Because gamma radiation does not elicit neutrons (i.e., the subatomic particles that can make substances radioactive), irradiated products and/or their packaging are not made radioactive. Regardless of the radiant energy source, the effect of ionizing energy on a product is the same. Energy penetrates the product and its packaging, but most of the energy simply passes through the product, leaving no residue. The small amount of energy that does not pass through the product is negligible, and is retained as heat.

Either gamma rays or e-beam systems typically are employed for industrial sterilization of liquid products, e.g., liquid pharmaceuticals and liquid medical, surgical, and research products. In terms of system operation and design, industrial sterilization by gamma rays and e-beam systems is carried out in a very similar manner. Various types of walls, shields, and mazes are used to prevent radiation from leaving the irradiation chamber. Outside the irradiation chamber, on the non-sterile side of the facility, product is loaded onto sterilization containers (also called “carriers” or “totes”). For gamma irradiation, the products are introduced into the cell area either manually in a batch system, or on a conveyor in a continuous loading system. The gamma radiation source is moved (e.g., mechanically) from its storage position into a raised operating position for irradiation. By comparison, the automatic conveyor system can be employed to transport loaded product into the irradiation chamber past either a cobalt 60 gamma ray source rack or e-beam accelerator. Generally, in order to maximize dose uniformity, the product passes the source in such a way that two opposing sides are exposed during the irradiation process. After passing through the radiation field for a specified time interval or at a specified speed, the product exits the irradiation chamber into the sterile side of the facility, where dosimeters attached to the sterilization container or product are read to verify that the radiation dose received by the products were within the maximum and minimum values specified (i.e., typically from about 25 kGy to about 40 kGy for liquid products intended for use in pharmaceutical, medical, research and biotech applications). When this is confirmed, the product is released for shipment to the customer (e.g., after any necessary sterility test is performed).

The absorbed dose delivered to the product is a function of the duration of the exposure of the product to the radiation source, density of the product itself, and the amount of energy to which the product is exposed (i.e., which itself is a function of the quantity of cobalt-60 isotope or other radiation source in the system, and the distance of the product from the radiation source). For such bulk sterilization of liquid products, it is important that uniform exposure of the product to the radiation source is achieved.

There are two types of cobalt-60 sterilization facilities for product sterilization. In the source-overlap type, the cobalt source rack is larger than the product carrier, while in the product-overlap configuration, the product carrier is larger than the source rack. Multiple layers of product typically are used in order to pack as much product as possible around the source to maximize the gamma utilization efficiency. Processing capacity can be increased as needed by adding cobalt capsules to the source rack. To shield the source when not in use and to perform system maintenance, the cobalt-60 is stored, e.g., in a water-filled pool.

There also are two types of systems used in e-beam sterilization of liquid products. Direct-current systems employ a constant high voltage to accelerate electrons through a high potential difference and are most effective for electron energies up to 5 MeV. Radiofrequency accelerators employ a time-varying electromagnetic field for acceleration and are generally more effective above 5 MeV. Both types of system are built with output beam power of from about 10 kW to about 100 kW. The volume of product processed is related to the power of the accelerator such that, e.g., a 100 kW accelerator can process 10 times more of the same product in a specified time than a 10 kW accelerator. Electron accelerator beams are either vertical or horizontal. A horizontal beam facilitates two-sided irradiation, which often is required to achieve thorough penetration of product and good dose uniformity. On the other hand, a vertical beam facilitates conveyor loading. A single-layer conveyor typically is used, and product carriers are positioned as closely together as possible in the radiation chamber to maximize beam utilization efficiency. Because electron dose rates are very high, it is particularly important for e-beam sterilization that the product moves swiftly and smoothly as it passes through the radiation field in order to minimize dose variation.

Industrial radiation sterilization thus is a continuous, automated process with only a single parameter, namely, time of exposure, to be controlled. Major advantages of sterilization by irradiation include the isothermal nature of the procedure, and the ability to carry out sterilization using the final package in which the product will be marketed. Presently, however, due to the manner in which the processing is carried out, irradiation of packages containing smaller volumes only (e.g., at most, 15 liters) can be obtained. Larger product volumes cannot be achieved with products sterilized by terminal irradiation, but must be combined under aseptic conditions into a sterile package following irradiation, and typically require further sterile filtration. This pooling of irradiated product necessarily introduces the potential for contamination of the final product.

Thus, there exists a need in the art for a method and means for the radiation sterilization of larger product volumes, e.g., larger amounts of liquid product pooled in a single container. There also exists a need for a means, e.g., a sterilization container, for use in such sterilization that will allow uniform exposure of the product to the radiation source, e.g., in an industrial batch sterilization process. Because there also is a requirement for transporting the products into and out of the irradiation chamber and around the radiation source, there further is a need for an easily movable and moderately sized sterilization container that can be employed for the sterilization of liquid products. These, and other objects and advantages, as well as additional inventive features, are provided by the present invention and will be apparent from the description of the invention provided herein.

### BRIEF SUMMARY

The present invention provides, among other things, sterilization containers, inner bags, and methods for the radiation sterilization of liquid products. In particular, the invention preferably provides sterilization containers for the sterilization of a pooled volume of liquid product greater than about fifteen liters, and methods for sterilization by irradiation of liquid products.

Additional features and variations of the invention will be apparent to those skilled in the art from the entirety of this disclosure, including the detailed description, and all such features are intended as aspects of the invention. Likewise, features of the invention described herein can be recombined into additional embodiments that also are intended as aspects of the inventions irrespective of whether the combination of features is specifically mentioned herein as an aspect or embodiment of the invention. Also, only limitations that are described herein as being critical to the invention should be viewed as such; variations of the invention lacking limitations that have not been described herein as critical are intended as aspects of the invention. In addition to the foregoing, the invention includes, as an additional aspect, all embodiments of the invention narrower in scope in any way than the variations specifically mentioned herein.

### BRIEF DESCRIPTION OF THE FIGURES

FIG. 1 is a perspective view of a preferred embodiment of the sterilization container of the present invention having an optional preferred sliding container lid. The figure shows the optional placement inside the sterilization container of a preferred embodiment of the inner bag containing the liquid to be sterilized. Symbols: 100, inner bag; 110, container base; 120, side wall; 130, front wall; 140, back wall; 150, bottom panel; 160, container lid; 190, lip; 200, container lid rim; 210, lid grooves; 270, inner bag sterile access port.

FIG. 2 is a perspective view of a preferred embodiment of the sterilization container of the present invention having an optional preferred sliding container lid, and a u-shaped recess opposite the opening of said container base that intrudes into the interior of the sterilization container. The figure shows the optional placement inside the sterilization container of a preferred embodiment of the inner bag containing the liquid to be sterilized. Symbols: 100, inner bag; 110, container base; 120, side wall; 130, front wall; 140, back wall; 150, bottom panel; 160, container lid; 170, longer subpanel; 180, shorter subpanel; 190, lip; 200, container lid rim; 270, inner bag sterile access port.

FIG. 3 is a cross-section taken through a plane connecting the front and back walls of the sterilization container depicted in FIG. 1. Symbols: 110, container base; 130, front wall; 140, back wall; 150, bottom panel; 160, container lid; 190, lip; 200, container lid rim; 210, lid grooves.

FIG. 4 is a side planar view of a stack of preferred sterilization containers of the present invention, wherein the stacking is done by means of a preferred container lid adapted to allow another sterilization container to nest securely on top of the container lid. Symbols: 110, container base; 120, side wall; 150, bottom panel; 160, container lid; 210, lid grooves.

FIG. 5 is a perspective view of a preferred embodiment of the inner bag of the present invention, showing optional attachments to the inner bag that allow liquid to be pumped out of or into the inner bag while maintaining sterility. Symbols: 220, inner bag side wall; 230, inner bag front wall; 240, inner bag back wall; 250, inner bag bottom panel; 260, inner bag top panel; 270, inner bag sterile access port; 280, flange; 290, pinch clamp; 300, tubing; 310, male tubing insert; 320, removable sealing cap; 330, sanitary fitting; 340, removable dustcover; 350, zip lock poly bag.

The detailed description and examples that follow are provided to enhance the understanding of the invention, but are not intended to limit the scope of the invention.

### DETAILED DESCRIPTION

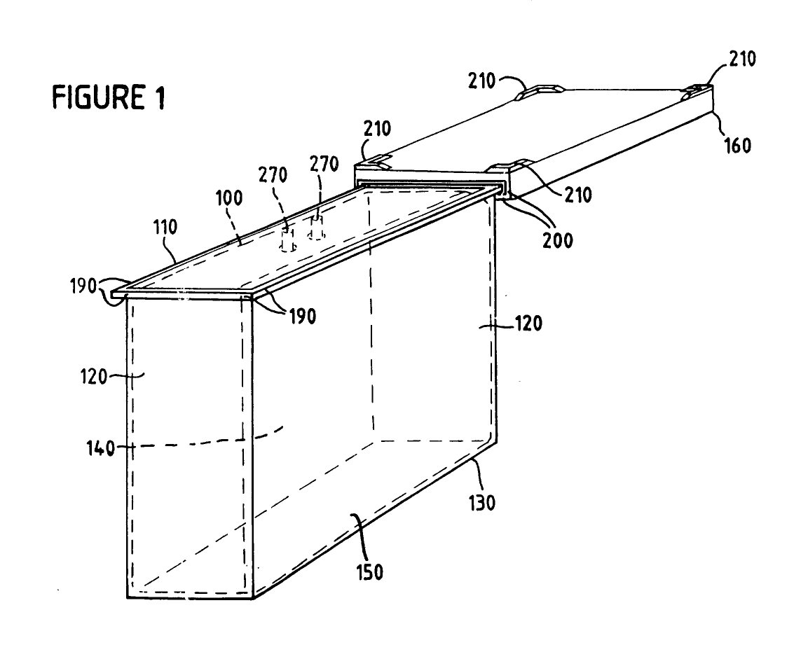

The present invention provides, among other things, methods for the sterilization by irradiation of liquid products, and preferred sterilization containers and inner bags for use in these methods. These elements are described separately below, with reference to FIGS. 1-5 containing the following numbered parts: 100, inner bag; 110, container base; 120, side wall; 130, front wall; 140, back wall; 150, bottom panel; 160, container lid; 170, longer subpanel; 180, shorter subpanel; 190, lip; 200, container lid rim; 210, lid grooves; 220, inner bag side wall; 230, inner bag front wall; 240, inner bag back wall; 250, inner bag bottom panel; 260, inner bag top panel; 270, inner bag sterile access port; 280, flange; 290, pinch clamp; 300, tubing; 310, male tubing insert; 320, removable sealing cap; 330, sanitary fitting; 340, removable dustcover; 350, zip lock poly bag.

Sterilization Containers

The present invention provides sterilization containers appropriate for sterilization of liquid products by radiation exposure, especially by gamma radiation exposure. The size and configuration of the sterilization containers as described herein optimizes the exposure of liquid product contained in the containers to the irradiation source.

In a preferred embodiment, a sterilization container comprises:

(a) a container lid; and

(b) a base comprising a bottom panel, and

extending from and connected to the bottom panel so as to be upstanding and circumferentially contiguous, and to terminate at a top end that registers with the container lid and defines an opening into an interior of the base:

(i) a first side wall and a second side wall; the first and second side walls being disposed opposite each other and spaced apart, and

(ii) a front wall and a back wall; the front and back walls being disposed opposite each other, spaced apart, and connected to the first side wall and the second side wall.

Preferably a sterilization container of the invention (unlike those known in the prior art) is appropriate for the irradiation sterilization of a relatively large pooled volume of liquid (i.e., “pooled” meaning combined in a single vessel such as an inner bag). In particular, preferably a sterilization container has a capacity of (i.e., an ability to contain) a volume greater than about fifteen liters, especially a volume greater than about 18 liters, and particularly a volume greater than about 25 liters, and thus is appropriate for the irradiation sterilization of such quantities: In particular, preferably a sterilization container has. a capacity of (and is appropriate for the irradiation sterilization of) a pooled volume of liquid (especially a pooled volume combined in one vessel) of from about 80 liters to about 120 liters, especially from about 90 liters to about 110 liters, and particularly about 80 liters, about 85 liters, about 90 liters, about 95 liters, about 100 liters, about 105 liters, about 110 liters, about 115 liters, or about 120 liters of liquid product.

A preferred embodiment of the sterilization container of the invention is depicted in FIG. 1. As can be seen from this figure, preferably the vessel containing the pooled volume of liquid, such as an inner bag 100, fits entirely within the interior of the container base 110. The “container base” comprises the sterilization container absent any container lid. The interior of the container base is created by the connection of the first and second side walls, front and back walls, and container bottom panel.

The present invention encompasses the preferred embodiment depicted in FIG. 1, and also encompasses sterilization containers that vary in size from this preferred embodiment. In particular, with reference to FIG. 1, preferably the container has inside dimensions of from about 10 to about 12 inches wide, from about 22 to about 37 inches long, and from about 22 to about 26 inches high, and a wall thickness of from about 0.25 to about 1 inch. Preferably the the width of the first and second side walls 120 can vary from about 10 to about 12 inches, preferably the length of the front and back walls 140 can vary from about 22 to about 37 inches, preferably the height of the side, front and back walls can vary from about 22 to about 26 inches, and preferably the thickness can vary from about 0.25 to about 1 inch.

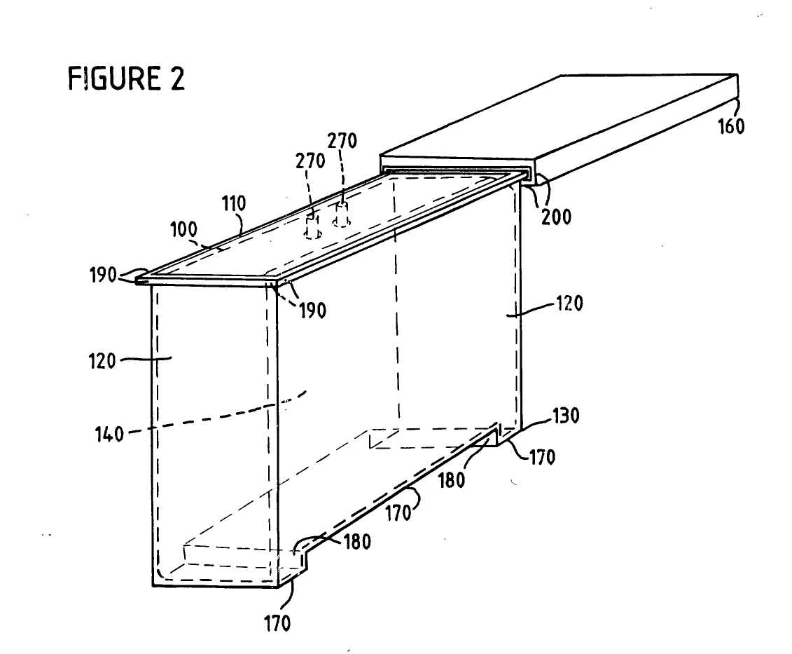

The length and width of the bottom panel 150 and container lid 160 (e.g, depicted in FIGS. 1 and 2) desirably can vary according to the container size. However, preferably the bottom panel, 150, has a length and a width that allow it to connect to, and together with the side, front and back walls, form the container base. Preferably the container lid, 160, has a length and a width that allows it to register with the opening into the interior of the container base.

Two criteria for modifying the size and configuration of the sterilization container according to the invention (e.g., the preferred embodiment depicted in FIG. 1) are that: (1) the side walls have a width that is appropriate for and allows relatively uniform sterilization by irradiation of a liquid product placed inside the irradiation chamber; and (2) for irradiation sterilization of liquid products such as described herein (e.g., industrial sterilization), preferably a total dose to the liquid product ranging from a particular minimum and maximum absorbed dose is obtained. For instance, the total dose to liquid product typically ranges from about 25 kGy to about 40 kGy, although for certain applications (e.g., products that need to meet more stringent European standards for sterilization) a total dose ranging from about 30 kGy to about 40 kGy is preferred, and for other certain applications (e.g., with radiation-resistant products) a total dose ranging from about 25 kGy to about 60 kGy may be acceptable. Accordingly, less variation is permitted in the length of the side walls of the sterilization container of the invention, and the product placed inside the sterilization container (e.g., pooled inside an inner bag) must fall within density constraints imposed by the dose range (which, in turn, impacts the size of the sterilization container).

In a particularly preferred embodiment according to the invention (e.g., with reference to FIG. 1), the sterilization container desirably has inside dimensions of from about 11 inches wide by about 23 inches long by about 25 inches high, and a wall thickness of about 0.5 inches. Preferably the first and second side walls 120 each have a width of about 11 inches, and a height of about 25 inches. Desirably, the front wall 130 and back wall 140 that are connected to the first and second side walls each have a length of about 23 inches and a height of about 25 inches.

In another preferred embodiment of the sterilization container of the invention, the bottom panel and front and back walls of the sterilization container desirably are adapted to allow the sterilization container to be easily transported, e.g., by loading the sterilization container onto a pallet, trolley or dolly, or onto any other portable platform for handling, storing, or moving materials and packages. Preferably the sterilization container is adapted to comprise at least one recess into the interior of the sterilization container, especially at least one u-shaped recess. Even more preferably, the sterilization container is adapted as depicted in FIG. 2 to comprise a u-shaped recess opposite the opening into the interior of the container base, intruding into the interior of the sterilization container. Optionally, the sterilization container can comprise one, two, or three such recesses which furthermore, can vary in their shape (e.g., can be rounded, or other than v-shaped).

With reference to this preferred sterilization container shown in FIG. 2, preferably the vessel inside the sterilization container containing the pooled volume of liquid, such as an inner bag 100, fits entirely within the interior of the container base 110. Preferably the sterilization container has inside dimensions of from about 11 inches wide by about 30 inches long by from about 22 to about 24.5 inches high, and a wall thickness of about 0.5 inches, and further accommodates the u-shaped recess. As depicted in FIG. 2, preferably the first and second side walls 120 have a width of about 11 inches, and a height of from about 22 to 24.5 inches. In particular, the height of different areas of the first and second side walls can vary to accomdate the recess into the interior of the sterilization container. Preferably, however, instead of the bottom panel being a single plane perpendicular to the sterilization container's side, front, and back walls (e.g., as depicted in FIG. 1), the bottom panel preferably is notched to comprise planes perpendicular to the sterilization container's side, front, and back walls, and planes parallel to the sterilization container's side walls (e.g., as depicted in FIG. 2). As depicted in FIG. 2, preferably in this embodiment the bottom panel comprises five separate yet connected subpanels, and that make up these parallel and perpendicular planes. Desirably, a total of three longer subpanels are the planes directed perpendicular to the sterilization container's side, front, and back walls, and each longer subpanel is separated by a total of two shorter subpanels. Preferably the two shorter subpanels are the planes directed parallel to the sterilization container's side walls. The shorter subpanels desirably range from about 2 to about 4 inches (preferably about 2.5 inches) in length. The longer subpanels can be the same or different lengths from each other, and are any length or combination of lengths so as to make up the length of the remainder of the bottom panel, and so as to to allow the bottom panel to be loaded onto a portable platform. In this embodiment the front wall 130 and back wall 140 also desirably have been adapted as depicted in FIG. 2 to accommodate the, u-shaped recess of the bottom panel. Namely, preferably the front and back wall each have a length for their majority of about 30 inches and a height of about 24.5 inches. However, on the bottom side of the front and back wall that connects with the bottom panel, preferably there is (e.g., centered) on the length a notch ranging from about 3 to about 27 inches in length, and from about 2 to about 4 inches (preferably about 2.5 inches) in width.

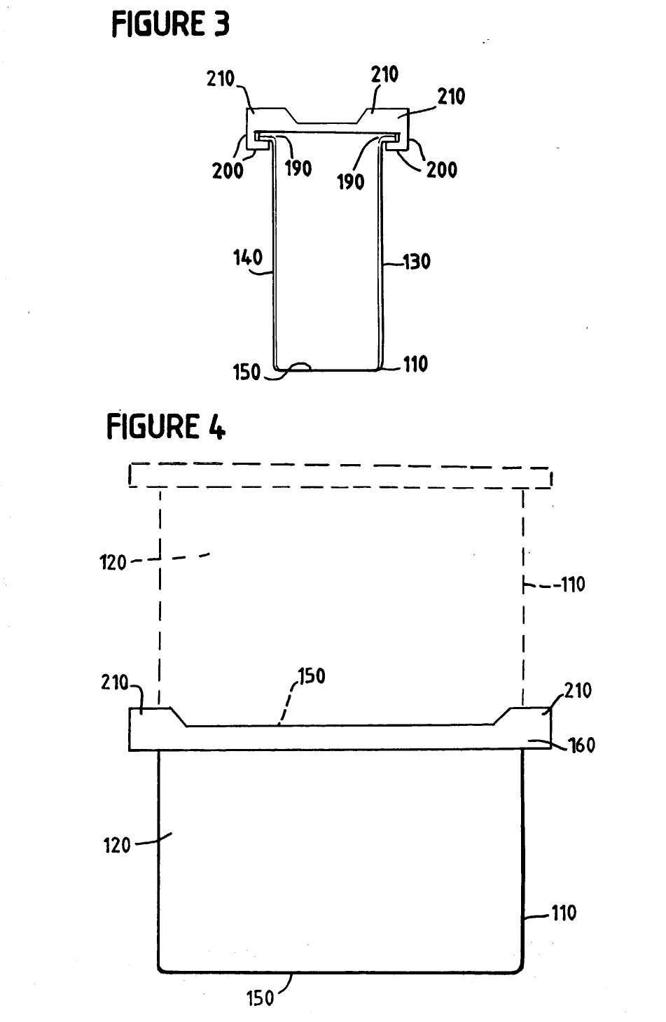

In yet another preferred embodiment of the invention (which also is depicted in FIGS. 1 and 2), the top end of each of the front wall and back wall of the sterilization container optionally comprises a lip 190 extending outward from the opening of the container base. Also, optionally, the top end of the first side wall and second side wall of the sterilization container comprises a lip 190 extending outward from the base container opening. Preferably the lip extends outward from about 0.5 to about 2 inches, optimally from about 1 to 2 inches, especially about 1.5 inch. In such an embodiment where one or more walls of the container base comprises a lip, preferably the container lid of the sterilization container has been adapted to comprise a container lid rim 200, depicted in cross section in FIG. 3. Preferably the sterilization container comprises a container lid rim that has an outwardly flared portion adapted to register to the lip of the container base when the container lid is installed on the sterilization container base. As depicted in FIG. 3, preferably the container lid installs on the container base by sliding the container lid rim over the lip. However, other means of installing the container lid on the container base can be employed according to the invention.

Optionally, as shown in cross section in FIG. 3, and by a front perspective view in FIG. 1, the container lid of the sterilization container can comprise lid grooves 210 on a surface opposite the container lid rim 200. Preferably the lid grooves are adapted to register with the bottom panel of a sterilization container base such that one container can be placed on another so as to nest on the container lid comprising the lid grooves and form a sterilization container stack. According to the invention, a stack can be as few as two sterilization containers nested series, and as many as ten sterilization containers. Preferably a stack comprises five sterilization containers nested in series. FIG. 4 shows a stack of preferred sterilization containers of the present invention, where the stacking is done by means of a preferred container lid adapted to allow another sterilization container to nest securely on top of the container lid by virtue of the container lid grooves 210. The stacking of the sterilization container thus maximizes use of floor space, e.g., for storage, and particularly in the irradiation chamber.

Also depicted in FIG. 4 is another preferred container lid 160 according to the invention. The alternate preferred container lid does not comprise any container lid rim, but instead, merely abuts flush against the container base. The container lid having the rim is preferred for use in applications where it might be necessary, for instance, to move the sterilization container from the uppermost part of a stack, e.g., by means of a fork lift or crane, and it is important that the sterilization container remain closed during this transport. In such an application, the outwardly flared container lid rim provides a means of gripping the sterilization container, and yet, due to the lid rim being securely slid over the lip of the container base, there is little or no opportunity for the sterilization container to open. It is for this reason also (i.e., additional security regarding the closure of the sterilization container) that it is preferred according to the invention that the lid rim slides over the lip at the top end of the front and back walls (i.e., as opposed to sliding over the lip at the top end of the side walls).

The sterilization containers of the invention (e.g., as depicted in FIG. 1 and FIG. 2), preferably have an overall size and configuration as described herein that allows for a total dose to liquid product contained therein ranging from a particular minimum and maximum absorbed dose. For instance, this total dose preferably ranges from about 25 kGy to about 40 kGy, although for certain applications (e.g., liquid products that need to meet more stringent European standards) a total dose ranging from about 30 kGy to about 40 kGy is preferred, and for other applications (e.g., with radiation-resistant products) a total dose ranging from about 25 kGy to about 60 kGy may be preferred. The dose to product preferably should not substantially change regardless of whether or not the irradiated sterilization container is present in a stack.

The sterilization containers of the invention preferably are designed to withstand the demands of industrial filling, transport, storage, and sterilization by irradiation, especially by gamma irradiation. Additionally, preferably the sterilization containers can be frozen, e.g., prior to, during and/or following irradiation of the liquid product present in the inner bag. For sterilization of liquid product that is serum, serum substitute, or plasma, preferably the inner bag containing the product is frozen inside the sterilization container prior to irradiation. Typically, however, the irradiation of the frozen product is done at ambient temperature. Accordingly, the sterilization containers preferably withstand variation in temperatures (e.g., variation such as a change from freezing, thawing, refrigeration, or storage at ambient temperature) as well as temperature extremes (e.g., particularly temperatures below freezing).

Accordingly, the walls of the sterilization containers, and optionally the container lid and the bottom panel of the sterilization containers, preferably are comprised of any material appropriate for sterilization by irradiation of liquid product placed inside the sterilization containers (e.g., liquid product pooled inside a vessel such as an inner bag placed in the sterilization containers). In particular, preferably the sterilization containers are comprised of stainless steel or plastic, or the following resins (or modified forms of these resins), e.g: high-density polyethylene; low-density polyethylene; nylon (polyamide); polycarbonate; polymethyl methacrylate (acrylic); polystyrene; polysulfone; polyurethane; Teflon TFE (tetrafluoroethylene); polyethylene (e.g., especially cross-linked high-density polyethylene). Preferably the sterilization containers comprise polyethylene, or a modified form of polyethylene, especially a low temperature modified polyethylene resin. The walls of the sterilization containers desirably can be either opaque, semi-opaque, or non-opaque. In a preferred embodiment, the walls of the sterilization containers are opaque.

Inner Bags

The sterilization containers of the invention preferably are used in conjunction with a vessel placed in the interior of the containers (e.g., an inner bag) which contains the pooled liquid product and optimizes the handling of the pooled liquid products, e.g., liquid biologicals. Preferably only a single inner bag is used in a sterilization container, although optionally, more than a single inner bag can be employed. The inner bag, when present, preferably is the vessel in which the finished liquid product is marketed. “Finished liquid product” is a liquid product that has undergone all stages of production, including terminal irradiation sterilization, and optionally, including labeling and/or addition of outer packaging (e.g., packaging placed over the inner bag).

Thus, the inner bags employed according to the invention desirably are designed to withstand the demands of industrial filling, handling, transport, storage, and sterilization by irradiation (e.g., especially by gamma irradiation). Additionally, preferably the inner bags can be frozen, e.g., prior to, during and/or following irradiation of the liquid product. For liquid product that is serum, serum substitute, or plasma, preferably the inner bags containing the product are frozen inside the sterilization containers prior to, and following irradiation, and optimally are maintained frozen (e.g., at about −10°C.) until such time as employed for use in a particular application. With such freezing, optionally serum sterilized according to the invention can be stored and maintain sterility for up to about 4 years. Typically, however, the irradiation of the frozen product is done at ambient temperature. Accordingly, the inner bags preferably are designed to withstand variation in temperatures (e.g., variation such as a change from freezing, thawing, refrigeration, or storage at ambient temperature), as well as temperature extremes (e.g., particularly temperatures below freezing).

Preferably the inner bags are sterile prior to their use in the sterilization methods of the invention (i.e., prior to the deposition in the inner bags of the liquid product to be sterilized by irradiation), and are able to withstand further radiation exposure sufficient to sterilize any liquid product they contain. The inner bags thus optimally are able to withstand at least two sterilizations by radiation in the event the initials sterilization of the bags is done by radiation sterilization. Desirably the inner bags (and product contained therein) are stable (e.g., remain sterile and intact) following at least about a six-to nine-month period of storage following terminal irradiation sterilization according to the invention. In particular, following terminal irradiation sterilization, desirably the inner bags do not discolor, generate odors, show altered chemical resistance, show altered melt temperature, or become more brittle, stiff, hard, or soft. Information regarding the selection of materials appropriate for packaging of products to be radiation sterilized is set forth in Hemmerich, “Polymer Materials Selection for Radiation Sterilized Products”, Medical Device and Diagnostic Industry (February 2000), (hereby incorporated by reference for its teachings regarding material selection), and other references well known to those skilled in the art.

An inner bag according to the invention thus preferably is comprised of at least one layer, and desirably is multi-layered. In particular, preferably the inner bag comprises from at least one to as many as ten layers, and even more desirably, comprises three, four, or five layers. The same resins (or modified forms of the resins) as employed for the sterilization containers of the invention also optionally can be employed for one or more layers of the inner bag, e.g.: high-density polyethylene; low-density polyethylene; nylon (polyamide); polycarbonate; polymethyl methacrylate (acrylic); polystyrene; polysulfone; polyurethane; Teflon TFE (tetrafluoroethylene); polyethylene (e.g., especially cross-linked high-density polyethylene). In particular, however, preferably an inner bag is comprised of at least three layers, with the first layer that contacts the liquid product preferably being a polymer resin (e.g., especially a film, for instance, a Q17 film), the second layer that contacts the first layer being an ultra high barrier co-extrusion, and the third layer being a polyethylene (e.g., a low-density polyethylene). Additionally, instead of there being only a third layer of polyethylene in the inner bag, the inner bag optionally can comprise a third, fourth, and fifth layer of polyethylene. In such an embodiment, preferably the third layer of polyethylene is modified to provide optimum performance at low temperatures, whereas the fourth layer of polyethylene desirably comprises a monolayer, high tensile low-density polyethylene, and the fifth layer of polyethylene optionally comprises a modified, puncture-resistant low-density polyethylene. Other arrangements and materials for the layers of the bag are well known to those skilled in the art. The inner bags (like the sterilization containers) preferably either can be opaque, semi-opaque, or non-opaque. To assist with visualization of the liquid product contained within the inner bag (e.g., such as where sterility following storage is visually confirmed), preferably an inner bag is non-opaque, and even more desirably is transparent. Preferred inner bags are those marketed by TC TECH Corporation (Minneapolis, Minn.). However, other appropriate inner bags including those by other vendors alternately can be employed.

Preferably an inner bag is designed to fit entirely (and optionally, relatively snugly) inside a sterilization container, especially when filled to capacity (e.g., filled to about 100%) or near capacity (e.g., filled to within from about 85% to about 99%, about 85%, about 86%, about 87%, about 88%, about 89%, about 90%, about 91%, about 92%, about 93%, about 94%, about 95%, about 96%, about 97%, about 98%, or about 99% of capacity). Preferably an inner bag is appropriate for sterilization of a relatively large volume of liquid, e.g., pooled in one inner bag. In particular, preferably an inner bag has a capacity (i.e., can hold) and is appropriate for sterilization of a volume greater than about fifteen liters, especially a volume greater than about 18 liters, and particularly a volume greater than about 25 liters. Preferably an inner bag is appropriate for sterilization (e g., in one inner bag) of from about 80 liters to about 120 liters, especially from about 90 liters to about 110 liters, and particularly about 80 liters, about 85 liters, about 90 liters, about 95 liters, about 100 liters, about 105 liters, about 110 liters, about 115 liters, or about 120 liters of liquid product.

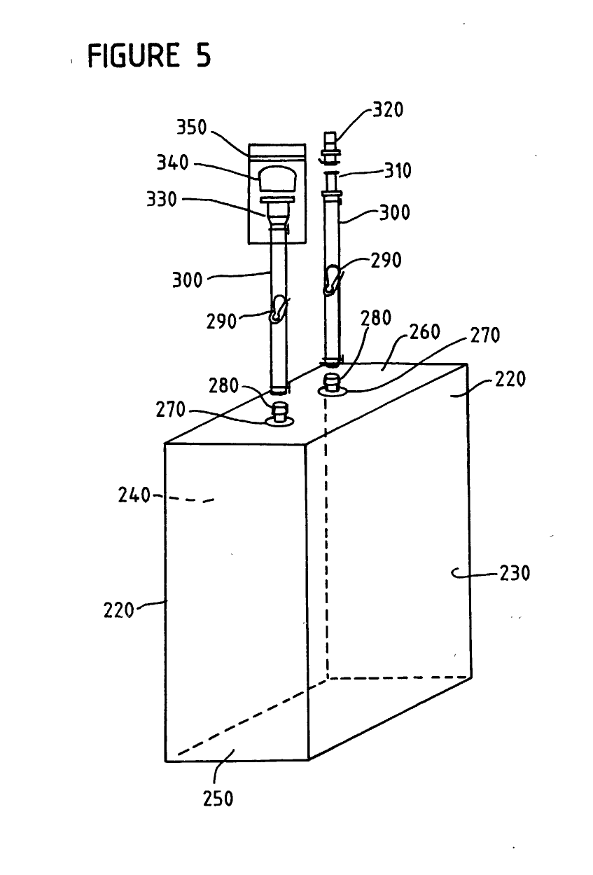

An inner bag according to the invention optimally has a sufficient size and shape that permits it to fit entirely (and optionally, relatively snugly) in a sterilization containing according to the invention. Thus, as depicted in FIG. 1, preferably an inner bag (especially an inner bag containing a pooled volume of liquid product) has inside dimension of from about 10 to about 12 inches wide, from about 22 to about 37 inches long, from about 22 to about 26 inches high. For reason of clarity, the inner bag absent the sterilization container is depicted in FIG. 5. As can be seen from FIG. 5, preferably the inner bag, like the sterile container itself, comprises inner bag first and second side walls 220 that have a width of from about 10 to about 12 inches, and a height of from about 22 to about 37 inches. Desirably, the inner bag front wall 230 and inner bag back wall 240 (that are connected to the inner bag first side wall and the second side wall) each have a length of from about 22 to about 37 inches and a height of from about 22 to about 26 inches. This size and configuration of the inner bag optimizes the placement of the inner bag in the sterilization container and exposure of liquid product contained therein to the irradiation source. Preferably, however, the width of the inner bag side walls is about 11 inches, the length of the inner bag front and back walls is about 23 inches, and the height of the side, front and back walls is about 25 inches. The inner bag thickness (i.e., the thickness of all the layers of a multilayer bag) can vary from about 1 to about 40 mil, and preferably is about 30 mil. The length and width of the inner bag bottom panel 250 and inner bag top panel 260 (e.g., depicted in FIG. 5) necessarily will vary with the inner bag size. Preferably the inner bag top and bottom panels have a length and a width that allow these panels to connect to the inner bag side, front and back walls.

In a preferred embodiment as depicted in FIG. 5, the inner bag optionally has one or more sterile access ports 270, for example, to allow quantities of a liquid to be placed within the inner bag or to be removed, e.g., by pump. The access port optionally is connected to a flange 280. The flange itself optionally is connected to a pinch clamp 290, which itself optionally is connected to tubing, 300. The tubing projecting from one access port according to the invention optionally is connected, e.g., by means of a male tubing insert 310, to a removable sealing cap 320. The tubing projecting from the other access port according to the invention optionally is connected, e.g., by means of a sanitary fitting 330, to a removable dustcover 340, which itself connects to a zip lock poly bag 350. Of course other attachments to the one or more access ports are contemplated according to the invention, and would be apparent to the ordinary skilled artisan.

Liquid Products

Liquid products present in a sterilization container of the invention preferably are contained (i.e., are pooled, or combined) within a vessel such as an inner bag placed in the sterilization container. The preferred sterilization containers and methods of sterilization according to the invention are appropriate for the sterilization of any liquid product that is not radiation sensitive (i.e., for sterilization of radiation-insensitive products), or for sterilization of any liquid product that is not deleteriously impacted by the particular amount of radiation employed for the sterilization process described herein (i.e., for sterilization of radiation level-insensitive products). Optimally, the absence of any deleterious effect on the liquid product by irradiation sterilization is confirmed experimentally.

Preferred liquid products for sterilization according to the invention are liquid products (e.g., liquid biologicals) employed in the pharmaceutical, research, and medical arenas, such as sera, vaccines, antibiotics, antimycotics, salt solutions (e.g., balanced salt solutions), enzyme solutions, media (e.g., buffered media and/or tissue culture media), and tissue transplant chemical reagents. Particularly preferred liquid products according to the invention are sera, sera substitutes and plasma, including, but not limited to, bovine sera, calf sera, donor calf sera, chicken sera, fetal bovine sera, guinea pig sera, horse sera (e.g., EIA-free—donor herd), fetal horse sera, lamb sera, newborn calf sera, porcine sera, rabbit sera, goat sera, mouse sera, cat sera, dog sera, sheep plasma, bovine embryonic fluid, and human sera. Other preferred products include other liquid biologicals (e.g., especially protein supplements and growth supplements). Particularly preferred liquid products for sterilization by the method of the invention are those marketed by Biologos, Inc. (Montgomery, Ill.).

The liquid products (e.g., like the sterilization containers and inner bags) either can be opaque, semi-opaque, or non-opaque. The liquid products optionally cat contain suspended or dispersible particulates, beads, anal the like, depending on the nature of the products.

Sterilization Methods

Among other things, the present invention provides preferred methods for sterilizing liquid products. In a preferred embodiment the method comprises:

(a) disposing the liquid product (e.g., especially pooled, or contained within one vessel such as an inner bag) in a sterilization container having an internal volume greater than about fifteen liters, and

(b) exposing the sterilization container to an amount of radiation sufficient to sterilize the liquid product. In particular, preferably the method comprises disposing the liquid product in a sterilization container according to the invention, and then exposing the container to the radiant energy source.

In terms of the amount of radiation sufficient to sterilize the liquid product, either a gamma ray irradiation system or an e-beam system can be employed in the method of sterilization according to the invention. Such irradiation systems are described in U.S. Pat. No. 6,051,185, Williams, “Weighing the Choices in Radiation Sterilization: Electron-Beam and Gamma”, Medical Device and Diagnostic Industry, 68-72 (March 1995), and Farrell et al., “Selecting a Radiation Sterilization Method”, Medical Device and Diagnostic Industry, 85-90 (August 1995) (each incorporated herein by reference). Preferably the irradiation system is an industrial irradiation system. According to the invention, however, irradiation by use of gamma rays is particularly preferred. The dose ranges described herein are given in units appropriate for gamma irradiation sterilization (e.g., preferably a total dose to liquid product ranging from about 25 kGy to about 40 kGy, especially from about 30 kGy to about 40 kGy, and optionally from about about 25 kGy to about 60 kGy). Such dose ranges are calibrated based on gamma irradiation sterilization, and can be converted to units appropriate for e-beam sterilization, as is well known in the art.

For gamma ray irradiation systems, the strength )f the cobalt-60 source is measured in units of megacurie (Mci). For the ⁶⁰Co Radioisotope, 1 Mci produces 14.8 kW of gamma rays. Industrial cobalt-60 sources for strerilization applications as described herein range in strength from about 1 to about 7 Mci, which corresponds to a power range of from about 15 to about 100 kW. Such sources typically are constructed as an array of “pencils” arranged in a thin vertical slab or plaque. Since a point gamma source emits isotopically, the optimum configuration with regard to dose uniformity and efficiency results when both the height and width of the source are as large as practical. Typical plaque sources measure from about 2 to about 3 m high by from about 3 to about 4 m wide. The dose rate near the gamma ray source is in the range of from about 1 to about 10 kGy/hr.

For e-beam irradiation systems, industrial electron accelerators used for sterilization applications as described herein range in power from about 10 to about 200 W. After electrons are accelerated to high energy in a pencil-like beam in a high-vacuum enclosure, they are magnetically scanned back and forth to expand the beam before it exits the vacuum into atmosphere (e.g., through a thin metal window). In this manner, the accelerator produces an intense, highly localized, unidirectional beam of electrons. The e-beam scan typically measures about 1 m long and from about 5 to 10 cm wide. The average dose rate in the uniform region of the scanned beam is between 2 and 50 kGy/sec.

Ultimately, with both gamma and e-beam irradiation systems, an absorbed radiation dose appears (at least nominally) as thermal energy in the product. A calorie is defined as the thermal energy required to raise the temperature of 1 g of water by 1° C. In gamma sterilization, the dose typically is delivered over such a long period of time (e.g., from about 4 to about 8 hours) that the temperature of the irradiation chamber and the product remain essentially equal. The radiant heat emitted from the cobalt source and the energy absorbed in the radiation shield determines the ambient temperature in the irradiation chamber. The result is that in many industrial gamma facilities, product temperatures can be expected to run from about 10° C. to about 20° C. above ambient temperature. For this, and other reasons (e.g., reasons related to product stability), it may be preferred that th liquid product disposed within the sterilization container (e.g., in an inner bag) is frozen prior to sterilization.

Preferably either during or following, the sterilization procedure, the radiation dose to products being irradiated (i.e., the total dose to product or “absorbed dose”) is measured. Dosimeters optionally can be used for this purpose. Dosimeters desirably are employed that give a quantitative measurement of the dose received by the product itself, independent of the dose rate. Preferably the dosimeters are inserted in a batch being sterilized in sufficient number to give an accurate assessment of the dose received by each product assessed. A “batch” is any number of liquid products being sterilized during a given cycle of sterilization. Optionally, samples from a batch can be further assessed for sterility. Preferably, any samples that are tested for sterility are from parts of a batch that are considered to be at most risk of contamination. The Food and Drug Administration's “Sterilization Process Validation Guidelines” (hereby incorporated by reference in its entirety for its teachings regarding sterility testing) provides information regarding methods of obtaining validation data for the irradiation process, as do other references that are well known to those skilled in the art.

“Sterilization” according to the invention means the successful inactivation in a liquid product of adventitious agents such as viable organisms (including, but not limited to, bacteria and fungi), viruses, or bacteriophages, or the reduction to an acceptable level of viable organisms, viruses, mycoplasma or bacteriophages. The “acceptable level” can vary with a particular application. For instance, the acceptable level of a particular organism might be a level to which humans or mammals can be exposed and yet not contract any infection or disease. Typically, however, the acceptable level is a level at which, using techniques available at the time of the invention, the particular viable organisms, viruses, or bacteriophages, cannot be detected in the liquid product. Tests for bacterial and fungal contamination are routine, and are well known in the art. For instance, such tests are carried out for aerobic and anaerobic contaminator, e.g., at around 26° C. or around 37° C. on a variety of different culture media (e.g., tryptose phosphate broth, tryptic soy broth, thioglycollate broth, blood agar plates, Sabouraud agar plates, and nutrient agar, to name but a few). Mycoplasma testing can be done using well known tests, e.g., using the method of Hayflick and the large volume inoculation technique of Barile. Tests for bacteriophages also are well known. Virus screening can be done, e.g., using standard virus tests. especially tests for the presence or absence of viruses using the direct fluorescence antibody technique.

The need for removal of viruses from liquid products, such as biological liquids being sterilized, is particularly acute. Biological liquids include liquids that are obtained from biological sources, for instance body liquids such as blood or fluids, or liquids derived from cell culturing, especially culturing of recombinant cells. All such biological liquids have a certain risk of being contaminated with infectious agents, especially viruses, which should not be present in the final sterilized product. The risk of transmission of viruses by biological liquids is known. There is comprehensive literature available dealing with the inactivation of infectious agents by various methods. These methods include the treatment of biological and pharmaceutical products with chemical substances (e.g., with detergents, solvents, etc., or combinations thereof), heating steps (e.g., heating in an aqueous solution in the presence of stabilizing agents, heating in the dry state and heating in the solid wet state) and physical methods (e.g., photoinactivation or nanofiltration). However, such biological liquids, particularly products of human or animal blood or plasma, typically are intended for therapeutic, prophylactic or diagnostic applications. Such products may contain proteins, such as enzymes, proenzymes, coagulation factors, enzyme inhibitors, immunoglobulins, albumin, plasminogen, fibrinogen and fibronectin. Accordingly, these useful therapeutic moieties in the biological liquid preferably remain intact following irradiation sterilization, whereas any viable organisms, viruses, or bacteriophages preferably are inactivated (i.e., rendered nonfunctional).

The sterilization methods according to the present invention also optionally provide a means of inactivating aggregates or monomers of prion proteins, which are proteins with a molecular mass of at least about 30 kDa. Prions are believed to cause scrapie and scrapie-related diseases.

Confirmation of the functioning or efficacy of liquid products sterilized using the methods and/or sterilization containers of the invention also can be performed. For instance, liquids can be tested following sterilization for pH and other characteristics of the liquid. Culture media and sera can be assessed for active growth promotion and absence of cytotoxic effects, e.g., by comparison against pre-tested control lots.

The invention will now be described with reference to the following Example. The following Example is by means of illustration, not limitation. Of course, variation of this Example in the spirit and scope of the invention is contemplated herein.

### EXAMPLE

This example describes experiments that confirm the utility of a preferred sterilization container according to the invention in the present inventive preferred method of sterilization by radiation.

For these studies, about 100 liters of serum were placed in a sterilization container having an inner bag, as depicted in FIG. 2. Three batches were sterilized using the sterilization process, with two of the batches containing 15 sterilization containers, and one of the batches containing 14 sterilization containers. The filled sterilization containers were transferred to Steris/Isomedix (Libertyville, Ill.) where batch process irradiation sterilization was carried out. Subsequent tests confirmed the performance of the serum as compared to serum that had been sterilized as per prior art procedures.

These results thus confirm that the present method of radiation sterilization and sterilization container for use in same can be employed to obtain sterile liquid products.

All the references cited herein are hereby incorporated in their entireties by reference.

While the present invention has been described in terms of specific embodiments, it is understood that variations and modifications will occur to those in the art, all of which are intended as aspects of the present invention. Other similar modifications should be apparent as well. Modifications of the sterilization container and method of sterilization can be made without parting from the spirit and scope of the invention. Accordingly, this invention includes all modifications encompassed within the spirit and scope of the invention.

What is claimed is:

1. A sterilization container comprising:
(a) a container lid; and
(b) a base comprising a bottom panel, and extending from and connected to said bottom panel so as to be upstanding and circumferentially contiguous, and to terminate at a top end that registers with said container lid and defines an opening into an interior of said base:
(i) a first side wall and a second side wall; said first and second side walls being disposed opposite each other and spaced apart, and
(ii) a front wall and a back wall; said front and back walls being disposed opposite each other, spaced apart, and connected to said first side wall and said second side wall, wherein said sterilization container has a capacity of a volume of liquid greater than about fifteen liters.

2. A sterilization container according to claim 1, wherein said capacity is for a volume greater than about 25 liters.

3. A sterilization container according to claim 1, wherein said capacity is for a volume of from about 80 to about 120 liters.

4. A sterilization container according to claim 1, wherein said sterilization container has inside dimensions of from about 10 to about 12 inches wide, from about 22 to about 37 inches long, and from about 22 to about 26 inches high, and a wall thickness of from about 0.25 to about 1 inch.

5. A sterilization container according to claim 1, wherein said sterilization container has inside dimensions of from about 11 inches wide by about 23 inches long by about 25 inches high.

6. A sterilization container according to claim 1, wherein said first and second side walls of said sterilization container each have a width of about 11 inches, and a height of about 25 inches, and wherein said front wall and back wall each have a length of about 23 inches and a height of about 25 inches.

7. A sterilization container according to claim 1, wherein said bottom panel and front and back walls of said sterilization container have been adapted to facilitate transport of said container.

8. A sterilization container according to claim 7, wherein said sterilization container comprises at least one u-shaped recess opposite the opening of said container base that intrudes into said interior of said sterilization container.

9. A sterilization container according to claim 7, wherein said sterilization container has inside dimensions of from about 11 inches wide by about 30 inches long by from about 22 to about 24.5 inches high.

10. A sterilization container according to claim 7, herein said first and second side walls of said sterilization container have a width of about 11 inches, and a height of from about 22 to about 24.5 inches.

11. A sterilization container according to claim 7, wherein said bottom panel comprises planes perpendicular to said sterilization container's side, front, and back walls, and planes parallel to said sterilization container's side walls.

12. A sterilization container according to claim 11, wherein said bottom panel comprises at least five separate yet connected subpanels.

13. A sterilization container according to claim 12, wherein three of said five subpanels are longer subpanels that are said planes directed perpendicular to said sterilization container's side, front, and back walls, and wherein each longer subpanel is separated by two of said five subpanels, which are shorter subpanels, and are said planes directed parallel to said sterilization container's side walls.

14. A sterilization container according to claim 13, wherein said shorter subpanels range from about 2 to about 4 inches in length.

15. A sterilization container according to claim 1, wherein said container lid comprises a length and a width that allows said container lid to register with said opening into said interior of said container base.

16. A sterilization container according to claim 1, wherein said container lid comprises a rim.

17. A sterilization container according to claim 6, wherein said top end of each of said front wall and back wall comprises a lip extending outward from said opening.

18. A sterilization container according to claim 17, further wherein said top end of each of said first side wall and second side wall comprises a tip extending outward from said opening.

19. A sterilization container according to claim 17, wherein said container lid rim has an outwardly flared portion adapted to register with said lip when said container lid is installed on said container base.

20. A sterilization container according to claim 19, wherein said container lid installs on said container base by sliding said container lid rim over said lip.

21. A sterilization container according to claim 1, wherein said container lid comprises lid grooves on a surface opposite said container lid rim.

22. A sterilization container stack formed by obtaining at least a first and a second sterilization container according to claim 21, and nesting a container base of said first sterilization container on said container lid of said second sterilization container.

23. A method for sterilizing a liquid product, wherein said method comprises:
(a) disposing said liquid product in a sterilization container having a capacity of a volume of liquid greater than about fifteen liters, and
(b) exposing said sterilization container to an amount of radiation sufficient to sterilize said liquid product.

24. The method according to claim 23, wherein said liquid product is contained within one inner bag that is present inside said sterilization container.

25. The method according to claim 23, wherein said liquid product is a liquid biological selected from the group consisting of sera, sera substitutes, plasma, vaccines, antibiotics, antimycotics, salt solutions, enzyme solutions, media, protein supplements, growth supplements, and tissue transplant chemical reagents.

26. The method according to claim 25, wherein said sera is selected from the group consisting of bovine sera, calf sera, donor calf sera, chicken sera, fetal bovine sera, guinea pig sera, horse sera, fetal horse sera, lamb sera, newborn calf sera, porcine sera, rabbit sera, goat sera, mouse sera, cat sera, dog sera, sheep plasma, bovine embryonic fluid, and human sera.

27. The method according to claim 23, wherein said liquid product is frozen prior to irradiation.

28. The method according to claim 23, wherein said radiation is gamma radiation.

29. The method according to claim 23, wherein said exposure to radiation is done to obtain a total dose to said liquid product ranging from about 25 kGy to about 40 kGy.

30. A method for sterilizing a liquid product, wherein said method comprises:
(a) disposing said liquid product in a sterilization container according to claim 1, and
(b) exposing said sterilization container to an amount of radiation sufficient to sterilize said liquid product.
